(19) United States
(12) Patent Application Publication
(10) Pub. No.: US 2011/0060224 A1
(43) Pub. Date: Mar. 10, 2011

(54) NON-INVASIVE CONTINUOUS DOPPLER MONITORING DEVICE FOR ARTERIAL BLOOD FLOW TO DISTAL BODY PARTS

(75) Inventor: Brad Eliot Kessler, San Diego, CA (US)

(73) Assignee: TZ MEDICAL, INC., Portland, OR (US)

(21) Appl. No.: 12/852,797

(22) Filed: Aug. 9, 2010

Related U.S. Application Data
(60) Provisional application No. 61/232,443, filed on Aug. 9, 2009.

(51) Int. Cl. A61B 8/06

(52) U.S. Cl. 600/454

(57) ABSTRACT

A Doppler sensor for non-invasive continuous Doppler monitoring for arterial blood flow to a distal body part of a patient that employs a Doppler transducer for placement at the distal body part of the patient and an adhesive conductive gel for placement between the Doppler transducer and the distal body part of the patient. The adhesive conductive gel holds the Doppler transducer in position at the distal body part of the patient during medical procures so that continuous monitoring of arterial blood flow is provided via a Doppler monitor.

This application claims priority of U.S. provisional patent application 61/232,433, filed Aug. 9, 2009, entitled NON-INVASIVE CONTINUOUS DOPPLER MONITORING DEVICE FOR ARTERIAL BLOOD FLOW TO DISTAL BODY PARTS

### BACKGROUND OF THE INVENTION

This invention relates to monitoring of blood flow, and more specifically to a non-invasive blood flow monitoring device and method.

Many times during certain medical procedures, blood flow is restricted to purposely decrease blood flow and create a wanted blood clot, i.e. during femoral sheath removal post PTCA/Stent. With such blood flow restriction, it is important to ensure that distal body parts are receiving adequate blood flow. For example, in some procedures, an upper leg portion will be compressed or clamped to reduce arterial blood flow to the leg, to minimize bleeding during the procedures or post procedure. However, some blood flow must be maintained to ensure adequate supply to the extremities, to avoid tissue damage.

To ensure adequate blood flow is occurring while the compression/clamp is applied, current practice is to intermittently apply a Doppler monitoring device at a pulse point of the relevant distal body part, to determine if there is sufficient blood flow. Typically the blood flow is monitored in the posterior tibial artery, dorsalis pedis artery, or radial artery. The monitoring device includes a hand-held probe (transducer) and monitor which provides an audible representation of blood flow. The probe consists of, for example, an elongate probe body such as a pencil-like steel tube, or other probe body with contoured shape and materials for more comfortable hand-held use by medical personnel, with a Doppler chip placed flush with one end of the probe body, and sensor wires running from the Doppler chip, up the center and out the other side of the probe body. The probe wires are connected to a Doppler monitor device which transmits signals to the Doppler chip and reads sensed return signals sent back to the monitor from chip, converting the signals to, for example, an audio signal which the medical personnel can hear to determine that blood flow is sufficient.

Doppler pulses are sometimes difficult to find when using the traditional Doppler device, even though the pulse is known to be present. With intermittent monitoring, an MD/RN/tech has to relocate the pulse each time the probe is applied (which can be more complicated since blood flow has been reduced or might be insufficient to provide a pulse), and the intervals of monitoring must be sufficiently closely spaced to avoid long periods of insufficient blood flow. If a surgical procedure is underway, timing of the monitoring check points may not be practical relative to the timing needs of the surgical procedure.

Since the monitoring is only done periodically, the status of blood flow between monitoring points is unknown, and a positive flow status during the monitoring times may not accurately reflect the blood flow state between monitoring points.

To attempt to address some of the difficulties, intermittent monitoring, medical personnel time requirements and requiring relocating a pulse each time, some medical teams have attempted to tape the monitor probe to the patient at the location where the pulse is found. However, since the probe is designed to be hand-held and moved around to be positioned in a given location only very briefly, the probe configuration and shape, with the transducer at the tip end of a generally elongate probe body, makes it difficult to securely position and maintain the probe in the proper location and orientation such that it can properly detect the blood flow over extended periods. Also, it is important that the efforts of positioning the sensor and monitoring not interfere with the medical procedure itself.

### SUMMARY OF THE INVENTION

In accordance with the disclosure a small, light, portable, easy to use/install device is provided so that a reduced number of personnel are required in medical procedures, with no or minimal interference with the medical procedure from the sensor configuration and attachment.

Accordingly, it is an objective of the present system and method to provide an improved blood flow monitoring system to recognize in a more timely manner, any interruption to distal blood flow before tissue damage occurs.

It is a further object of the present system and method to enable medical staff the opportunity to focus on more immediate patient needs (vital signs changes, patient presentation) instead of spending time searching for pulse on either the upper or lower extremities.

It is yet another objective of the present system and method to provide an improved system and method for continuous arterial blood flow monitoring at distal body parts.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements.

### BRIEF DESCRIPTION OF THE DRAWINGS

FIG. 1 is a top view schematically illustrating use of the system and method;

FIG. 2 is a perspective view of a particular embodiment of the sensor in accordance with the system and method; and

FIG. 3 is an exploded perspective view of the sensor of FIG. 2.

### DETAILED DESCRIPTION

The system and method according to a preferred embodiment comprises a Doppler transducer with adhesive for attachment to a patient and connecting wires to connect to a Doppler monitor.

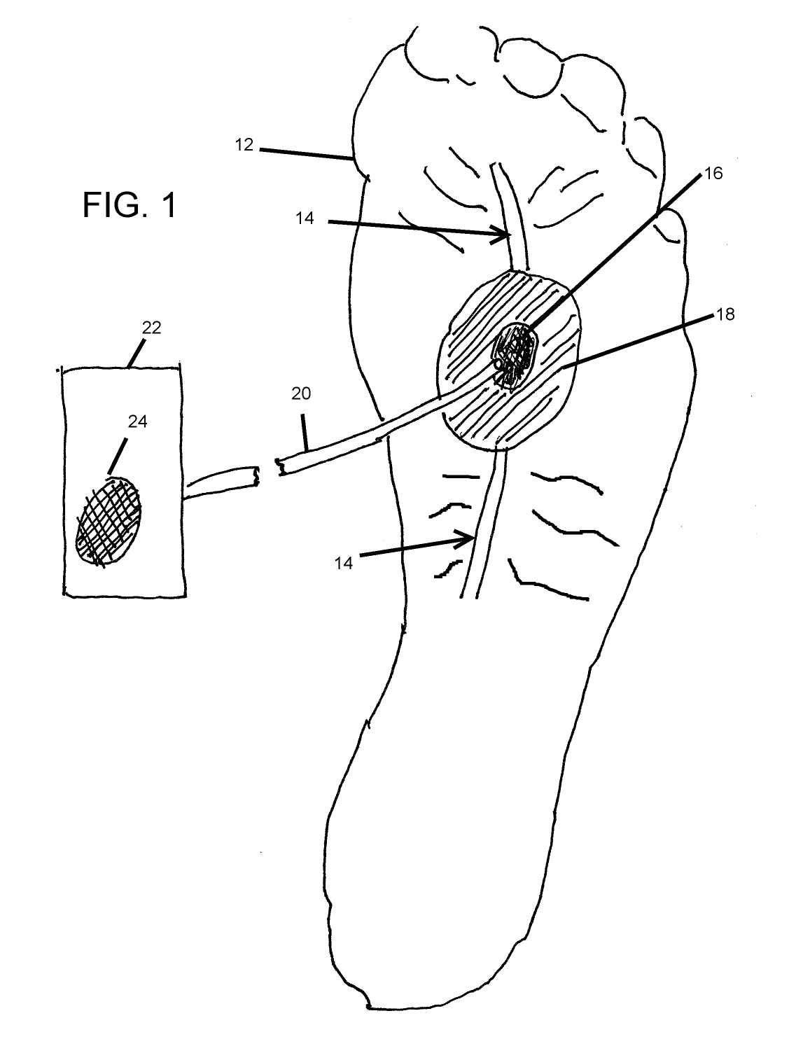

Referring to FIG. 1, a top view schematically illustrating use of the system and method, a patient's foot 12 is shown, with the location of the dorsalis pedis artery 14 visible. A Doppler transducer 16 is positioned on the outside of the foot, maintained in position until later removal by use of adhesive conductive gel 18. Cable 20 provides a connection between Doppler monitor 22 and the Doppler transducer 16, such that the monitor can interact with the transducer to produce audio, via speaker 24, representing sensed blood flow.

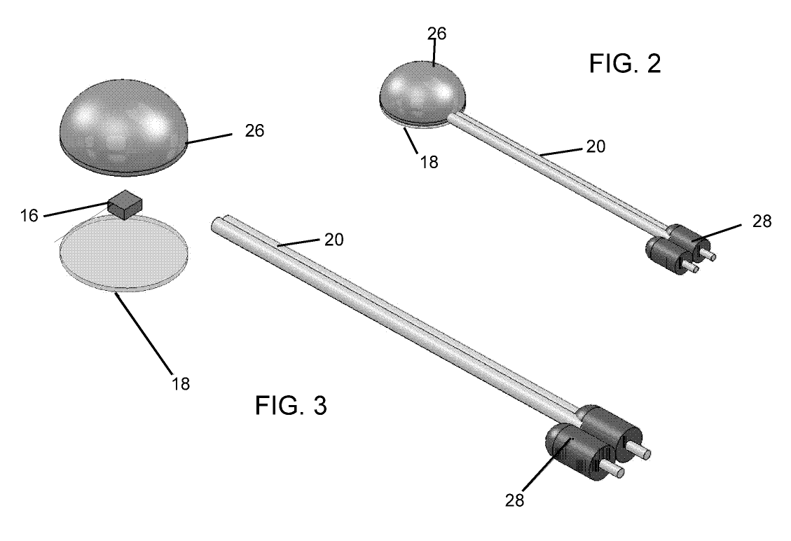

FIG. 2 is a perspective view of a particular embodiment of the sensor in accordance with the system and method, wherein a semispherical sensor overmold 26 is provided to contain the transducer therein and carry the adhesive for attachment to the patient. Adhesive conductive gel 18 is positioned on the bottom of the sensor overmold, and sensor cable 20 extends from the Doppler transducer 16 (not visible in FIG. 2) to a distal end carrying connectors 28, which comprise standard Doppler monitor connectors suitable to be received by a Doppler monitor.

Referring to FIG. 3, an exploded perspective view of the sensor of FIG. 2, the relative positions of sensor overmold 26, Doppler sensor 16 and adhesive conductive gel 18 are observed. In the illustrated embodiment, adhesive conductive gel 18 is a circular disk so as to be substantially co-extensive with the patient-side face portion of sensor overmold. In alternative embodiments, the adhesive conductive gel can be of different shape, or may be applied to the patient-side face portion separately prior to use.

Transducer 16 may comprise a piezoelectric ultrasonic transducer such as a model UZ250 sensor by Noliac (Noliac A/S of Denmark). Adhesive conductive gel 18 is suitably an adhesive conductive gel for attachment of the sensor to the patient, while enabling conduction of the Doppler signals from and to the transducer 16, and can comprise AG501 dermal fastener gel by AmGel Technologies of Fallbrook, Calif., or other adhesive compatible with Doppler signal transmission. Overmold 26 is suitably a EPDM (ethylene-propylene-diene monomer) polymer with a low durometer (e.g., 30 to 50). Exemplary materials for the overmold are provided under trade names Dutral brand Ethylene Propylene Copolymer by Polimeri Europa, Nordel brand by The Dow Chemical Company, and Vistalon brand by Exxon Mobil Corporation, for example. Connectors 28 are suitably standard AV connectors appropriate to be received by the Doppler monitor 22.

The overmold 26 may be provided in various shapes and configurations so as to fit or conform to the shape of the patient at the particular attachment location. The preferred and illustrated embodiment employs a semispherical shaped overmold 26. Other shapes, such as half egg-shaped may be employed. Also, the patient-side face is flat in the illustrated embodiment, but may also be contoured to conform more readily to the shape of the patient's body at a desired application position. All components that touch the patient are biocompatible.

Suitable dimensions of the sensor in particular embodiments are, 1 inch diameter circular disc or puck configuration, or, in the configuration shown in FIGS. 2 and 3, a 1 inch by inch semicircular dome having a dome with a inch height and having a 1 inch diameter at the base of the dome, the bottom face of the dome being a flat surface. The dome in FIGS. 2 and 3 is suitably radially symmetrical.

Procedures in which the device is useful include:

1) Arterial Sheath removal post Cardiac PTCA/Stent intervention while performing Manual Pressure or with the use of the Femstop Device.

2) Peripheral PTA/Stent intervention

3) Invasive lines (A-lines) including the use of IABP, Impella, LVAD, or CPS

4) Peripheral Surgery/procedures: Endarterectomy, Aorto/femoral bypass, Fem/Fem bypass, Fem/pop bypass.

In use, the device can be provided in a sterile package, which the medical personnel will open, to remove the device. Adhesive conductive gel 18 will have a removable protective covering thereover, such as a wax liner or the like, for protection prior to use. The covering is removed, and the exposed face of the gel is positioned on the patient at an appropriate location to enable sensing of blood flow. Connectors 28 are attached to a Doppler monitor 22, for continuous monitoring during the medical procedure. Once monitoring is no longer desired, the sensor portion is removed from the patient. The device may be provided in a single use pre-sterilized configuration, intended to be disposed of after use, or in a reusable configuration with adhesive replacement components provided for each use.

The illustrated use in FIG. 1 is on the patient's foot and dorsalis pedis artery, but other locations are also suitably monitored with the system and method, such as the leg and posterior tibial artery, or the arm and radial artery.

Accordingly, a device comprising a small, compact, transducer assembly which is easily attached to the body is provided, to enable continuous blood flow monitoring via Doppler monitor at distal body parts over extended periods of time. Continuous Doppler monitoring enables medical personnel to ensure that blood flow is occurring at the distal body parts to verify that excessive pressure or no newly formed blood clots are occurring within the artery during a procedure. This is all accomplished in accordance with the system and method without requiring the MD/RN/tech to intermittently apply a Doppler device and constantly have to relocate the pulse.

While a preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

What is claimed is:

1. A sensor module for non-invasive continuous Doppler monitoring for arterial blood flow to a distal body part of a patient, comprising:
a Doppler transducer for placement at the distal body part of the patient; and
an adhesive conductive gel for placement between the Doppler transducer and the distal body part of the patient for holding the Doppler transducer in position at the distal body part of the patient.

2. The sensor according to claim 1, further comprising a cable connecting to said Doppler transducer to carry signals to and from said Doppler transducer.

3. The sensor according to claim 1, further comprising an overmold cover holding said Doppler transducer therein.

4. The sensor according to claim 3, wherein said overmold cover is semispherical.

5. The sensor according to claim 3, wherein said overmold cover has a patient-side face for placement toward the patient's distal body part, and wherein said adhesive conductive gel is positioned between the patient and said patient-side face.

6. The sensor according to claim 5, wherein said patient-side face is substantially flat.

7. The sensor according to claim 5, wherein said patient-side face is contoured to conform to a shape of the distal body part of the patient.

8. A method for non-invasive continuous Doppler monitoring for arterial blood flow to a distal body part of a patient, comprising:
providing a Doppler transducer for placement at the distal body part of the patient;
providing an adhesive conductive gel for placement between the Doppler transducer and the distal body part of the patient for holding the Doppler transducer in position at the distal body part of the patient; and
connecting said Doppler transducer to a Doppler monitor device for detecting arterial blood flow.

9. The method according to claim 8, further comprising providing an overmold cover holding said Doppler transducer therein.

10. The method according to claim 9, wherein said overmold cover is semispherical.

11. The method according to claim 9, wherein said overmold cover has a patient-side face for placement toward the patient's distal body part, and wherein said adhesive conductive gel is positioned between the patient and said patient-side face.

12. The method according to claim 11, wherein said patient-side face is substantially flat.

13. The method according to claim 11, wherein said patient-side face is contoured to conform to a shape of the distal body part of the patient.

14. A system for monitoring for arterial blood flow to a distal body part of a patient, comprising:
a Doppler transducer for placement at the distal body part of the patient;
a Doppler monitor for providing signals to and receiving signals from said Doppler transducer and providing an output representing arterial blood flow; and
an adhesive conductive gel for placement between the Doppler transducer and the distal body part of the patient for holding the Doppler transducer in position at the distal body part of the patient.

15. The system according to claim 14, wherein said output comprises an audible output.

16. The system according to claim 14, further comprising a cable connecting said Doppler transducer to said Doppler monitor to carry signals to and from said Doppler monitor and Doppler transducer.

17. The system according to claim 14, further comprising an overmold cover holding said Doppler transducer therein.

18. The system according to claim 17, wherein said overmold cover is semispherical.

19. The system according to claim 17, wherein said overmold cover has a patient-side face for placement toward the patient's distal body part, and wherein said adhesive conductive gel is positioned between the patient and said patient-side face.

20. The sensor according to claim 20, wherein said patient-side face is substantially flat.

21. The sensor according to claim 20, wherein said patient-side face is contoured to conform more readily to the shape of the body part of the patient at a desired application position.
